(19) United States
(12) Patent Application Publication
(10) Pub. No.: US 2011/0184366 A1
(43) Pub. Date: Jul. 28, 2011

(54) Highly absorbent pad with integrity and durability

(75) Inventors: Raquel M. Carbonari, Philadelphia, PA (US); Jehann A. Biggs, Chicago, IL (US); Frank S. Glaug, Chester Springs, PA (US)

(73) Assignee: SCA HYGIENE PRODUCTS AB, Goteborg (SE)

(21) Appl. No.: 12/929,226

(22) Filed: Jan. 10, 2011

Related U.S. Application Data
(60) Provisional application No. 61/299,243, filed on Jan. 28, 2010.

(51) Int. Cl. A61F 13/53

(52) U.S. Cl. 604/372

(57) ABSTRACT

An absorbent article with improved integrity and durability includes a back sheet, a top sheet, and an absorbent core therebetween. The absorbent core passes a tumbling test for products having a total absorption capacity of between 260 g and 800 g, and passes a core resiliency test such that the article has a thickness when compressed laterally and released of less than 7 mm for an article having a total absorption capacity of 260-399 g and 11 mm for an article having a total absorption capacity of 400 g to 800 g. The absorbent core may be airlaid and include two or more layers made from pulp, superabsorbent polymer and/or bicomponent fibers. The various layers and materials create a complex shape retention network. As such, an improved liquid handling structure is created that distributes liquid evenly while maintaining its shape/thinness.

### CROSS-REFERENCE TO PRIOR APPLICATION

This application claims priority to U.S. Provisional Application No. 61/299,243 filed Jan. 28, 2010, which is incorporated by reference herein.

### BACKGROUND OF THE INVENTION

Absorbent articles for absorption of body fluids are available in a number of different designs. For absorption of urine and excrement, use is generally made of diapers or incontinence guards. There are other types of absorbent articles, such as feminine hygiene products. These products typically include a top sheet facing the body of the user, a back sheet facing the garment of a user, and an absorbent layer sandwiched between the inner, top sheet and outer, back sheet.

With prior art hygiene products, the absorbent core has been made of bulky fluff pulp material. As such, there has been a movement towards the use of thinner, higher SAP content cores. However, a drawback to these types of cores is a lack of integrity. In some situations, the typical mat formed absorbent cores are totally replaced by highly compressed air laid structures. For example, U.S. Patent Publication No. 2005/0045296 describes an airlaid composite core with pulp, superabsorbent material and bicomponent binder fibers.

However, one drawback to using airlaids in moderate to heavy absorbency incontinence pads is that these pads tend to be very stiff when the basis weight is increased and the thickness is decreased. In addition, because airlaids tend to be thinner and more compact, airlaid cores have sometimes a higher liquid penetration and absorption time. Accordingly, there is a need in the art for a material that allows for improved liquid distribution, yet also allows the material to retain its shape/thinness while being pliable.

### SUMMARY

According to a first aspect of the invention, there is provided an absorbent article comprising a back sheet, a top sheet, and an absorbent core therebetween. The absorbent core passes a tumbling test, and the article has a thickness when compressed laterally and released in a core resiliency test of less than 7 mm for an article having a Rothwell value from about 260 to about 399 g.

According to a second aspect of the invention, there is provided an absorbent article comprising a back sheet, a top sheet, and an absorbent core therebetween. The absorbent core passes a tumbling test and the article has a thickness when compressed laterally and released in a core resiliency test of less than 11 mm for an article having a Rothwell value from about 400 to about 800 g.

According to a third aspect, there is provided an absorbent article comprising a back sheet, a top sheet, and an absorbent core therebetween. The absorbent core is an airlaid material including two or more layers, and is made from a superabsorbent polymer, pulp, and bicomponent fibers, wherein the bicomponent fibers are disposed adjacent a top surface and bottom surface of the absorbent core.

A further feature of the first and second aspects is that the absorbent core is made of an airlaid material.

Yet a further feature of the first and second aspects is that the airlaid material includes two or more layers.

Yet a further feature of the second aspect is that the article has a Rothwell value from about 400 to about 600 g.

Yet a further feature of the first, second and third aspects is that the airlaid material comprises 0-10% by weight of a tissue or nonwoven layer, 20-65% by weight of pulp, 30-60% by weight of a superabsorbent polymer, 0-5% by weight polymer dispersion binder, and 2-10% by weight of bicomponent fibers.

Yet a further feature of the first, second and third aspects is that the airlaid material comprises 2.5%-4.5% by weight the tissue or nonwoven layer, 35-55% by weight of pulp, 40-50% by weight of the superabsorbent polymer, 1-3% by weight polymer dispersion binder, and 4-7% by weight of bicomponent fibers.

Yet a further feature of the first, second and third aspects is that the airlaid material comprises 40-50% by weight of pulp.

Yet a further feature of the first, second and third aspects is that a top surface and a bottom surface of the airlaid material is treated with a polymer dispersion binder.

Yet a further feature of the first, second and third aspects is that the absorbent core is an airlaid having a first layer, a second layer, and a third layer and the second layer is interposed between the first and third layers, the first and third layer being made from pulp and bicomponent fibers, and optionally superabsorbent polymers, the second layer is made from pulp and superabsorbent polymer.

Yet a further feature of the first, second and third aspects is that the first, third and an optional fourth layer contain bicomponent fibers, and optionally a polymer dispersion binder.

Yet a further feature of the first, second and third aspects is that the absorbent core includes an inner line and an outer line on each side of a longitudinal centerline, each the inner lines and outer lines including a plurality of slits having spaces therebetween.

Yet a further feature of the first, second and third aspects is that one or both of the outer line and the inner line are shaped as crescents.

Yet a further feature of the first, second and third aspects is that the absorbent article is an incontinence pad or a sanitary napkin.

### BRIEF DESCRIPTION OF THE DRAWINGS

The patent or application file contains at least one drawing executed in color. Copies of this patent or patent application publication with color drawings will be provided by the Office upon request and payment of the necessary fee.

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a perspective view of an exemplary absorbent article according to the features of the present invention.

FIG. 2 illustrates an exploded view of an exemplary absorbent article according to the features of the present invention.

FIG. 3 is a schematic showing a cross section of an exemplary absorbent article according to the features of the present invention.

FIG. 4 is a cross sectional view of an airlaid absorbent core according to features of the present invention.

FIG. 5 is a perspective view of a base structure of a dosing apparatus used to test features of the present invention.

FIG. 6 is a perspective view of a funnel of the dosing apparatus used to test features of the present invention

FIG. 7 is a perspective view of the dosing apparatus used to test features of the present invention

FIG. 8 is a side elevational view of the clamping apparatus used to test features of the present invention.

FIG. 9 is a top plan view of the clamping apparatus used to test features of the present invention.

FIG. 10 is a top plan view of the absorbent article secured by one clamp according to testing methods described herein.

FIG. 11 is a top plan view of the absorbent article secured by two clamps according to testing methods described herein.

FIG. 12 is a perspective view of the dosing apparatus positioned on an absorbent article between side walls of the clamping apparatus.

FIG. 13 is a perspective view of the absorbent article in a clamped position.

FIG. 14 is a perspective view of the absorbent article after being released from the clamping apparatus for measurement of the thickness of the absorbent article.

FIG. 15 is a perspective view of the dosing apparatus on the absorbent article.

FIG. 16 is a photograph of an absorbent article according to the prior art showing cracks after being run in a pilling box.

FIG. 17 is a photograph of an absorbent article according to the prior art showing cracks after being run in a pilling box.

FIG. 18 is a photograph of an absorbent article according to the prior art showing cracks after being run in a pilling box.

FIG. 19 is a photograph of an absorbent article according to the prior art showing cracks after being run in a pilling box.

FIG. 20 is a photograph of an absorbent article according to the prior art showing cracks after being run in a pilling box.

FIG. 21 is a photograph of an absorbent article according to features of the invention having no cracks after being run in a pilling box.

FIG. 22 is a photograph of an absorbent article according to features of the invention having no cracks after being run in a pilling box.

FIG. 23 is a photograph of an absorbent article according to features of the invention having no cracks after being run in a pilling box.

### DETAILED DESCRIPTION OF THE PREFERRED EMBODIMENTS

The present invention pertains to an absorbent article. An absorbent article is defined as an article or garment used for the absorption of body fluids, including but not limited to, infant diapers and training pants, adult incontinence products, feminine hygiene products, gender specific absorbent products, and pet training absorbent articles. While the preferred embodiment illustrates an incontinence pad, it should be understood that the present invention pertains to all types absorbent articles.

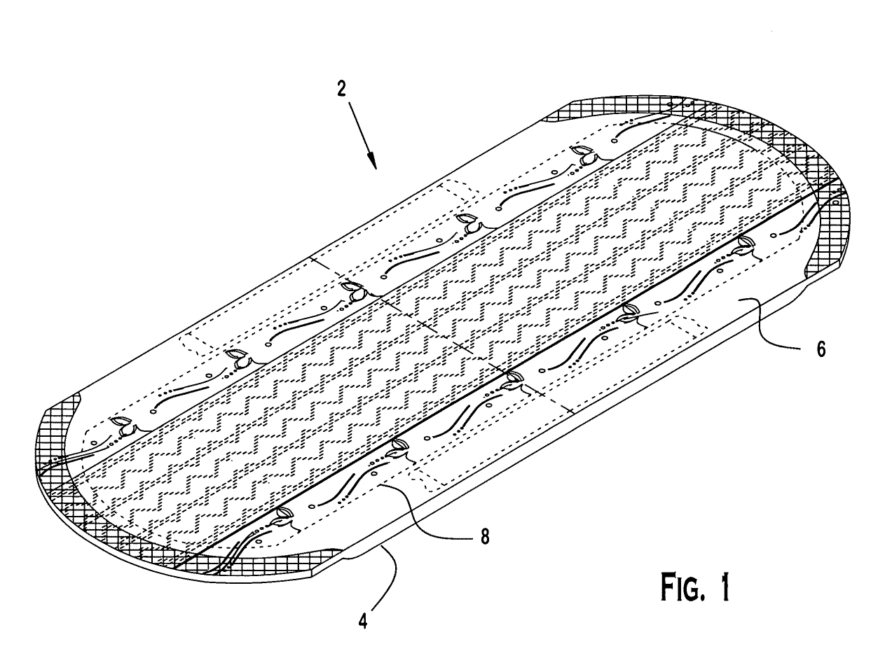

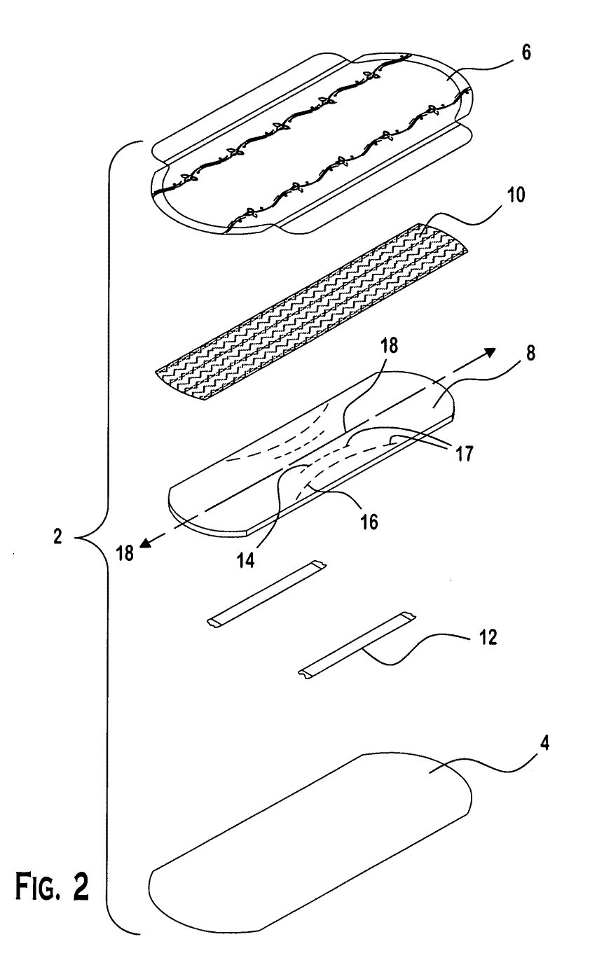

With reference to FIGS. 1 and 2, an absorbent article 2 includes a back sheet 4 and a top sheet 6 which sandwich an absorbent core 8 therebetween. The top sheet 6 can be joined together with the back sheet 4 and the absorbent core 8, for example by means of adhesive, ultrasonic joining or by means of some form of thermal bonding.

Preferably, the absorbent core 8 is appropriately manufactured from a suitable fiber material in the form of natural or synthetic fibers having absorbent properties, or a mixture of natural fibers and synthetic fibers or other absorbent materials of a previously disclosed kind that are suitable for use in sanitary towels, incontinence pads and panty liners, for example.

The absorbent core 8 may also contain a predetermined proportion of superabsorbent polymers (SAP). SAP materials are in the form of particles, fibers, flakes or similar, and have the capacity to absorb and to chemically bind liquid equivalent to several times their own weight while forming an aqueous gel. This provides a very high water-absorbent capacity in the finished product. The absorbent core 8 can exhibit different forms, for example an essentially elongated and rectangular form, or alternatively some other more irregular form, for example hourglass or triangular form.

Preferably, the absorbent core 8 includes 30-90% SAP, with a basis weight of about between 350 to 1000 g/m², and most preferably between about 400 to 1000 g/m². The absorbent core 8 may have a thickness of between about 2 mm to 10 mm and a density of between about 0.04 g/cm³ to 0.50 g/cm³.

In addition, the airlaid may be perforated according to the features described in U.S. Ser. No. 12/695,840, the entire disclose of which is incorporated by reference herein. For example, as shown in FIG. 2, the absorbent core 8 may include an inner line 14 and an outer line 16 on each side of a longitudinal centerline 18, each of the inner lines 14 and outer lines 16 including a plurality of slits 17 therebetween.

In addition, the absorbent core 8 may be made from two or more layers of material. For example, with reference to FIG. 4, the absorbent core 8 may include a first layer 24, a second layer 26, a third layer 28, and a tissue or other air permeable or nonwoven 30. Preferably, the first layer 24 and third layer 28 are made from pulp, bicomponent fibers, and optionally SAP. The bicomponent fibers may be made of polyethylene and polyethylene terephthalate (PE/PET). The bicomponent fibers act as a bonding agent, giving the airlaid its integrity, especially in the wet state. The second layer 26 is preferably made from pulp and SAP, and preferably does not include bicomponent fibers. A tissue or other air permeable or nonwoven layer 30 may also be included which acts as a carrier during the manufacturing process. However, any number of layers and materials may be used depending on application and design preference.

In addition, a polymer dispersion binder may be added to the top surface 32 and bottom surface 34 of the absorbent core 8. The polymer dispersion binder helps reduce dust and fiber loss. The polymer dispersion binder may be, for example, a latex binder. The various layers and materials create a complex shape retention network. As such, an improved liquid handling structure is created that distributes liquid evenly and therefore an even swelling of the superabsorbent polymer. This in turn allows the material to retain its shape/thinness while still delivering in absorbency.

Preferably, the airlaid material is made from 0-10% by weight of a tissue or nonwoven layer, most preferably about 2.5-4.5% by weight of a tissue or nonwoven layer, 20-65% by weight pulp, more preferably 35-55% by weight pulp, and most preferably 40-50% by weight pulp (preferably either treated or untreated cellulose), 30-60% by weight of a superabsorbent polymer, and most preferably about 40-50% by weight of superabsorbent polymer, 0-5% by weight polymer dispersion binder, and most preferably 1-3% by weight polymer dispersion binder, and 2-10% by weight bicomponent fibers, and most preferably 4-7% by weight of bicomponent fibers. It should be understood that all weight % refer to weight of the airlaid material.

The liquid-permeable top sheet 6 may include one or more layers of one or more of the following materials: a fibrous material, for example a soft nonwoven material, plastic film, mesh, open-celled foam, material laminate, etc. The top sheet 6 may include a perforated plastic film, for example, a thermoplastic plastic material such as polyethylene or polypropylene, or a mesh-like layer of synthetic or textile material. Synthetic mono-, bi-, or multi-component fibers, made of polymers such as polyethylene, polypropylene, polyester, nylon or the like, are preferably used as a nonwoven material. Also, natural fibers can be used in the top sheet, examples of natural fibers are cotton, regenerated cellulose, such as rayon, viscose, and silk. Mixtures of different types of fibers can also be used for the aforementioned nonwoven material.

The back sheet 4 is preferably liquid-impermeable (or at least possesses high resistance to penetration by liquid) and is thus so arranged as to prevent any leakage of excreted fluid from the product. The back sheet 4 may also be vapor-permeable. The back sheet 4 may be manufactured from a liquid-impermeable material which includes a thin and liquid-proof plastic film. For example, plastic films of polyethylene, polypropylene or polyester can be used for this purpose. Alternatively, a laminate of nonwoven and plastic film or other suitable layers of material can be used as a liquid-proof back sheet 4. In a previously disclosed manner, the clothing side of the back sheet 4 can be provided with beads of adhesive or some other previously disclosed attachment means, which can then be utilized for the application of the product to an item of clothing.

The absorbent article can also be provided with wings, that is to say folding flaps which are arranged along the sides of the product and can be utilized in conjunction with the application of the product.

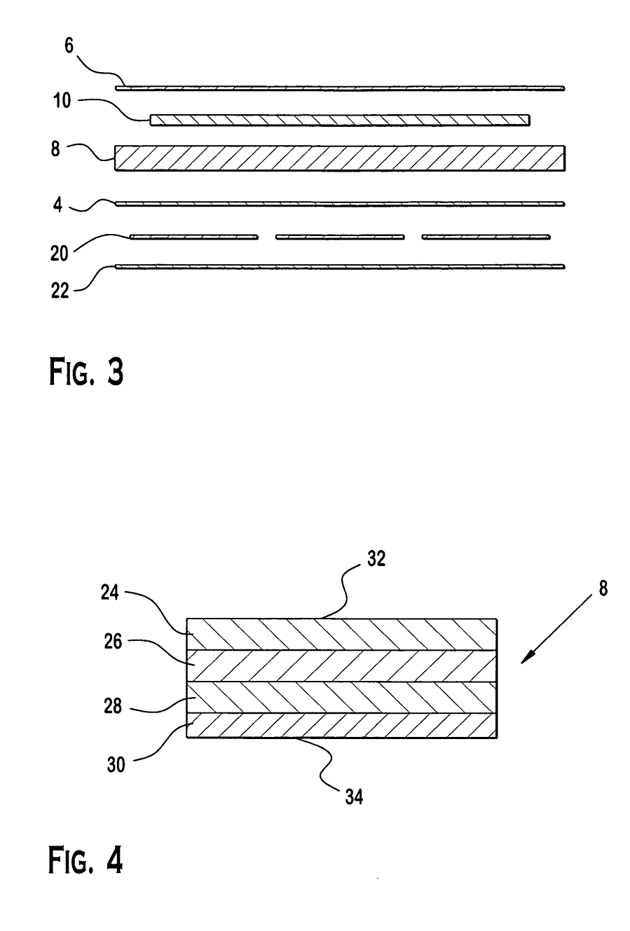

With reference to FIGS. 2 and 3, the layers of an exemplary embodiment of the absorbent article 2 of the present invention are illustrated. In particular, an acquisition/distribution layer 10 may be disposed between the top sheet 6 and the absorbent core 8, and elastics 12 (not shown in FIG. 3) may be preferably disposed between the top sheet 6 and the back sheet 4 along longitudinal sides of the absorbent article 2.

The acquisition/distribution layer 10 may be in the form of a wadding material having an appropriately specified thickness and resilience. The acquisition/distribution layer 10 may be made materials other than wadding material, such as an airlaid material, high loft material, or perforated films. The acquisition/distribution layer 10 may also incorporate fibrous materials in order to impart an appropriately balanced rigidity to it. The acquisition/distribution layer 10 can also incorporate an appropriate quantity of thermoplastic fibers in order to permit ultrasonic welding.

For feminine hygiene products and incontinence pads, an adhesive 20 may be provided on the garment facing side of the back sheet 4 for securing the absorbent article 2 onto a garment of a user. A release paper 22 may be provided adjacent the adhesive 20 layer.

The absorbent article 2 may be made from numerous other layers, as is known in the art. However, the specific layers used may vary, depending on design preference and application. For example, other layers not shown may be used, such as an additional back sheet, or any other intervening layer, such as SMS (Spunbond MeltBlown Spunbond), SSS (three layers of Spunbond), Carded NW, Hydroentangled, and the like. Likewise, the particular number and order of layers is optional, depending on application and design preference.

The absorbent article 2 of the present application has improved properties over the prior art absorbent articles. The following testing methods were used to ascertain the greatly improved core integrity over prior art absorbent articles.

### Inventive Product 1—Moderate

The product has, starting from the user facing side, a topsheet, and acquisition/distribution layer below the topsheet followed by an airlaid core and finally at the clothing facing side a backsheet. The topsheet is made of a carded nonwoven from Suominen, Finland having a basis weight of 23 g/m². The acquisition/distribution layer is made of a through air bonded nonwoven called Paratherm Loft 211 from TWE Dierdorf, having a basis weight of 50 g/m². The core is a three layered core built on a fourth tissue layer. The core is supplied from Glatfelter Falkenhagen GmbH in Germany. The first layer, facing the user side of the product is made of pulp, bicomponent fibres, superabsorbent particles and a latex binder. The second layer is made of pulp and superabsorbent material. The third layer is made of pulp, bicomponent fibres and a latex binder. The fourth layer closest to the backsheet is a tissue layer. The core has a basis weight of 460 g/m². The core is provided with crescent formed rows of slits in the crotch area. There are two rows on each side of the longitudinal centre line. These kinds of crescents made of slits are described in copending application U.S. Ser. No. 12/395,840. The backsheet is made of a polyethylene film called DH-284 PE Microflex from Clopay having a basis weight of 21 g/m². The different layers are glued together.

### Inventive Product 2—Heavy

The product has, starting from the user facing side, a topsheet, and acquisition/distribution layer below the topsheet followed by an airlaid core and finally at the clothing facing side a backsheet. The topsheet is made of a carded nonwoven from Suominen, Finland having a basis weight of 23 g/m². The acquisition/distribution layer is made of a through air bonded nonwoven called Paratherm Loft 211 from TWE Dierdorf, having a basis weight of 50 g/m². The core is a three layered core built on a fourth tissue layer. The core is supplied from Glatfelter Falkenhagen GmbH in Germany. The first layer, facing the user side of the product is made of pulp, bicomponent fibres, superabsorbent particles and a latex binder. The second layer is made of pulp and superabsorbent material. The third layer is made of pulp, bicomponent fibres and a latex binder. The fourth layer closest to the backsheet is a tissue layer. The core has a basis weight of 600 g/m². The core is provided with crescent formed rows of slits in the crotch area. There are two rows on each side of the longitudinal centre line. These kinds of crescents made of slits are described in copending application U.S. Ser. No. 12/395,840. The backsheet is made of a polyethylene film called DH-284 PE Microflex from Clopay having a basis weight of 21 g/m². The different layers are glued together.

### Core Resiliency Test

A core resiliency test of the present invention determines the absorbent core resiliency by way of vertical side compression. Essentially, an absorbent article is dosed with a certain amount of liquid. Thereafter, vertical side compression is applied, and then released. The thickness of the product after release is measured. A lower thickness value correlates to an absorbent core having an improved resiliency. The core resiliency test was generated to simulate the compression between the legs on an absorbent product. It is used to see how much the product deforms, and then returns to its original position.

The core resiliency test described herein is most useful for absorbent articles having a Rothwell value range of between about 260 g to 800 g. A Rothwell value is defined as the total absorption capacity as determined by the International Standard ISO 11948-1:1996(E).

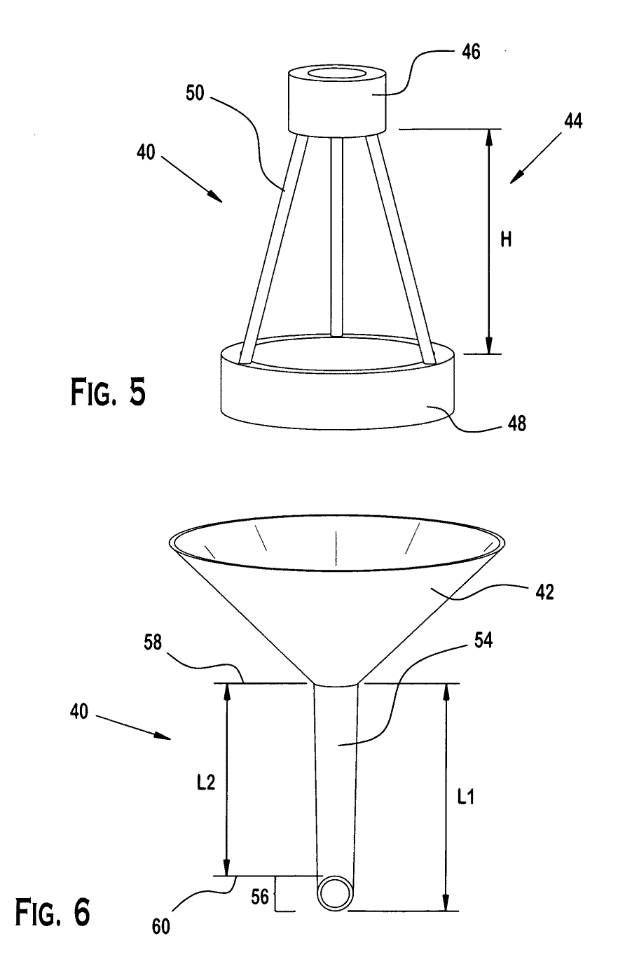

With reference to FIGS. 5-7, a dosing apparatus 40 includes a funnel 42 (shown in FIGS. 6 and 7) and a base support 44 (shown in FIGS. 5 and 7). As shown in FIG. 7, the base structure 44 supports the funnel 42 at a distance above the top surface of an absorbent article (not shown) that is placed underneath the base support 44 with the user side facing up.

As shown in FIG. 5, the base support 44 includes an upper hollowed cylindrical support 46 and a lower hollowed cylindrical support 48. The supports 46 and 48 are preferably made from stainless steel, and are separated by a distance H of about 45 mm. The upper hollowed cylindrical support 46 has an inner diameter of about 12 mm, an outer diameter of about 20 mm and a height of about 14 mm. The lower hollowed cylindrical support 48 has an inner diameter of about 46 mm, an outer diameter of about 54 mm, and a height of about 14 mm. Three cylindrical rods 50, also made from metal, connect the supports 46 and 48 and are spaced equidistant about the circumference of the supports 46 and 48. That is, the rods 50 are placed at 0°, 120°, and at 240°. Each rod 50 is preferably about 48 mm long. The weight of the dosing stand is about 115-125 g.

With reference to FIG. 6, the dosing apparatus 40 includes a funnel 42. The funnel may be either glass or plastic with a capacity of about 100 ml with a +/−5% weight tolerance. The funnel 42 preferably has an inner top diameter of about 61 mm and an outer top diameter of about 66 mm. The funnel 42 includes a dosing tube 54 at its lower end to funnel the liquid to a predetermined location on the absorbent article. The dosing tip 56 of the dosing tube is preferably cut at a 45° angle, so that a total length L1 of the dosing tube is preferably about 64 mm and the length L2 of the tube from its transition point 58 to the beginning of the cut portion 60 of the dosing tip 56 is about 55 mm. The dosing tip 56 preferably has an inner diameter of approximately 11 mm. Preferably, the distance between the end of the dosing edge of the funnel and the absorbent article should be between about 10-20 mm.

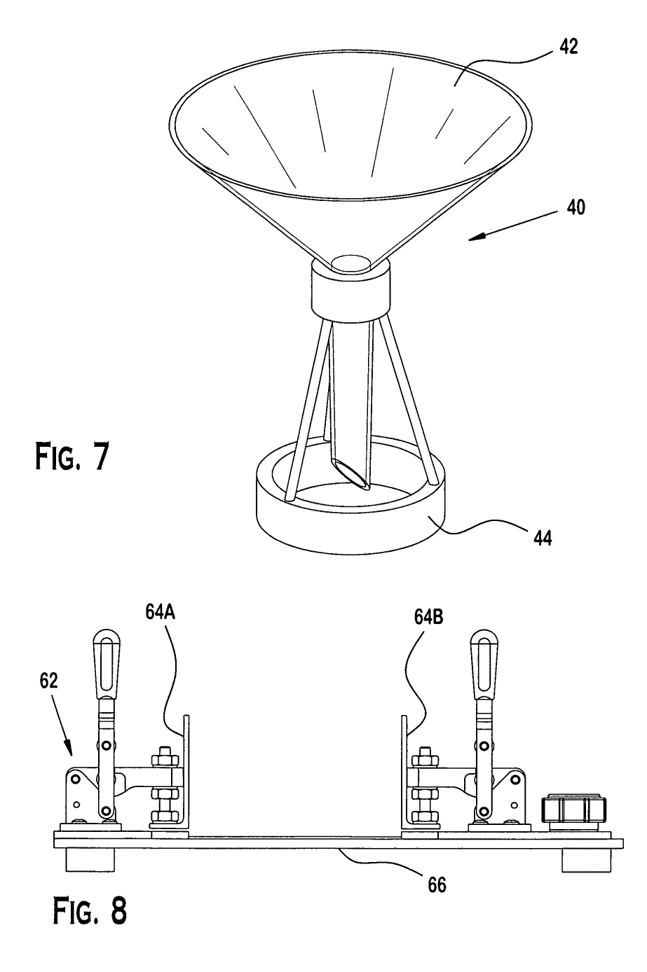

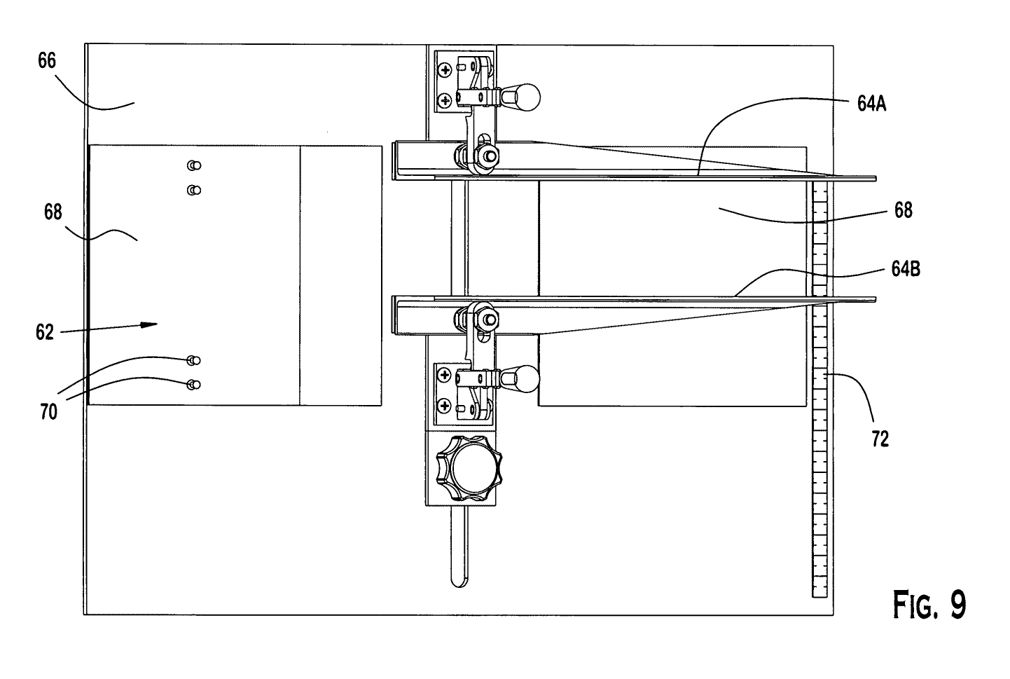

With reference to FIGS. 8-12, the clamping apparatus 62 of the core resiliency test will be described in more detail. The clamping apparatus 62 includes a pair of clamps 64A and 64B, as shown in FIGS. 8 and 9. The clamp 64A is stationary, while the clamp 64B is movable. The movable clamp 64B has the ability to slide and be fixed securely in place. The clamps 64A and 64B are preferably made of stainless steel, but may be made of other rigid and durable materials.

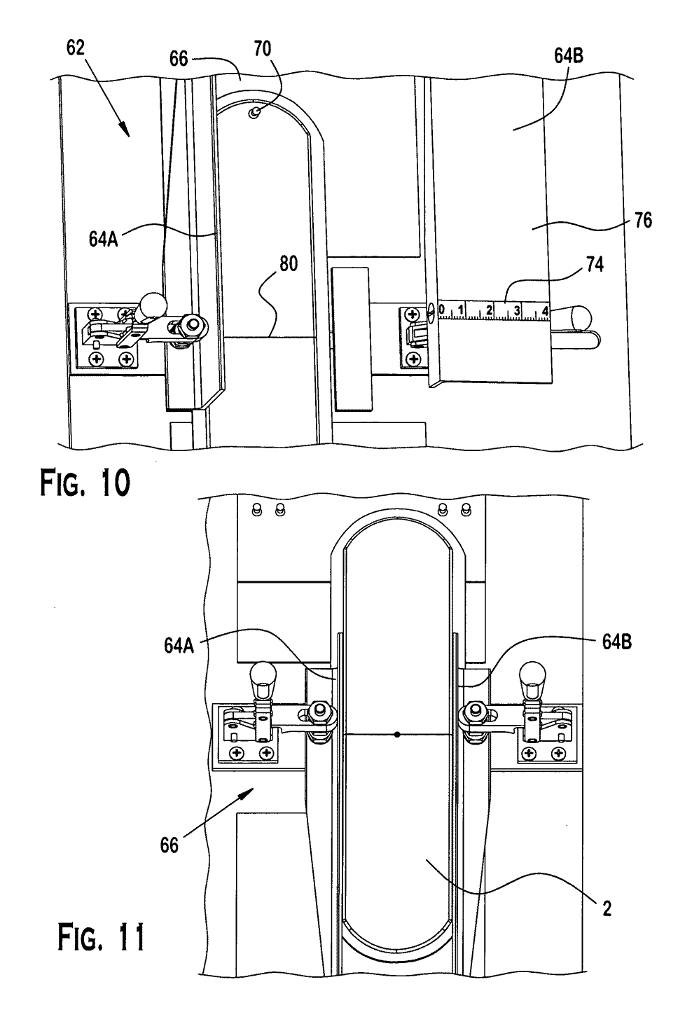

As shown in FIGS. 8 and 9, the clamps 64A and 64B are secured on a clamp base 66. Preferably, the clamp base 66 is made from plastic (but may be made of other materials), and has a dimension of about 300 mm×400 mm. However, other dimensions are possible, so long as the absorbent article fits thereon. In addition, at least two pieces of cork board 68 may be attached to the clamp base 66 so that the absorbent article may be secured on the clamping apparatus by pins 70 (see FIG. 9), or the like, during the mounting procedure. A clamp base ruler 72 may be disposed along one edge of the clamp base 66 so that the width of the absorbent article may be measured, as shown in FIG. 9. In addition, clamp rulers 74 are placed on the side vertical surfaces 76 of the clamps 64A and 64B (see FIGS. 10 and 11), so that the thickness of the absorbent article may be measured before and after the testing.

Prior to running the core resiliency test described herein, the specimens should be conditioned to room temperature of about (23+/−2° C) with a relative humidity of about (50+/−2%) for at least 24 hours. During the preparation stage, the absorbent articles should be removed from their bags and individual wrappings. Once the specimens are properly conditioned, release paper should be removed from the specimen (if present) and baby powder spread over the adhesive to remove its tackiness, and to prevent the specimen from sticking onto itself during the compression period.

After conditioning, the absorbent articles should be assessed. If an absorbent article includes a crack, an unpurposeful partial split/break, or is missing a section of its absorbent core, the product should be rejected. In total, thirty absorbent articles shall be tested per product variant. The absorbent articles should look identical taking into consideration minor process variations. If there is an obvious difference, the outlier should be rejected and replaced by another sample.

Once the absorbent articles are properly prepared, they are ready for testing. The first step is to measure the length and width of the absorbent article 2 so that the center of the product can be marked. Preferably, the absorbent articles' center line 80 is marked using a permanent marker, as shown for example in FIG. 10. That is, absorbent article 2 is marked along its width at the center of the absorbent article. Preferably, the thickness of the center line 80 should be between about 2-5 mm in width.

With reference to FIG. 10, the absorbent article 2 is mounted on the clamping apparatus 62 so that the user side of the absorbent article 2 is facing up. A first side of the article is clamped using the stationary clamp 64A. A pin 70 may be used to secure the absorbent article 2 onto the clamp base 66 while lowering the stationary clamp 64A onto the absorbent article 2. Preferably, the clamp 64A should cover approximately 10 mm of material, or sufficient material such that the pad is secured and the edge of the clamp is in side contact with the absorbent core. However, the absorbent core should not be clamped in the active clamp area during the initial clamping stage.

With further reference to FIG. 10, the center line 80 of the absorbent article 2 is centered on the clamp base 66. The movable clamp 64B is then moved and secured to the other side of the absorbent article, as shown in FIG. 11. Preferably, the clamp 64B should cover approximately 10 mm of material, or sufficient material such that the pad is secured and the edge of the clamp is in side contact with the absorbent core. However, the absorbent core should not be clamped in the active clamp area during the initial mounting stage.

With reference to FIG. 12, the dosing apparatus 40 is placed on top of the absorbent article 2 so that the tip 56 of the dosing tube 54 is directly above the center of the absorbent article 2 at a distance of between about 10-20 mm. Once the dosing apparatus 40 is placed on the top sheet, any wrinkles on the top sheet should be smoothed out. The absorbent article 2 is dosed with the appropriate dosing amount. In the present application, the absorbent articles 2 are dosed with about 75 ml of 0.9% saline solution (at room temperature, i.e., about 23+/−2° C.) for products with an average Rothwell value range of 260 g-800 g.

The fluid should be dosed at a substantially constant and relatively slow flow rate. That is, the dosing liquid should not overflow the lower hollowed cylindrical support 48 of the dosing apparatus 40, and the dosing liquid should all be absorbed by the absorbent article 2 in less than 1 minute. Once all the liquid has been absorbed, the dosing apparatus 40 is removed from the absorbent article 2. Using a timer, the absorbent article is allowed to rest for precisely 2 minutes.

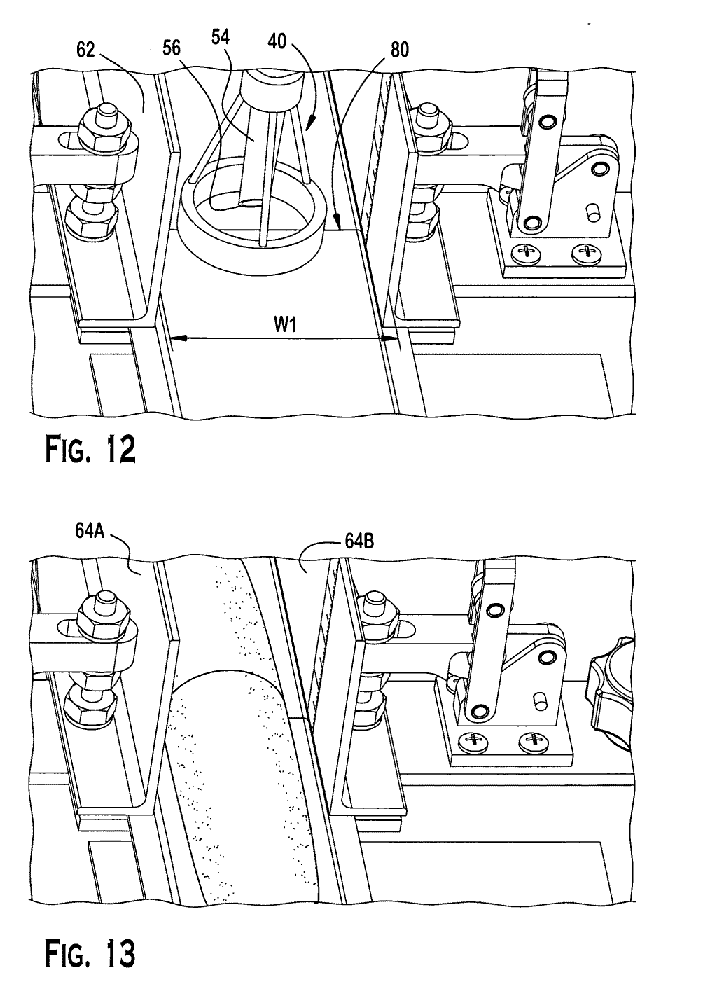

Once the product is dosed, the width W1 of the absorbent article 2 in the active clamping area is measured (see FIG. 12). The movable clamp 64B is then slid towards the stationary clamp 64A at a distance of (W1)/2 rounded down to the nearest whole number. Once the clamp 64B is slid into position, it is secured at this location (see FIG. 13).

Once the clamp 64B is secured in its clamping position, the absorbent article remains clamped for two minutes. After two minutes has passed, the clamp 64B is disengaged from its holding position by creating a small gap (i.e. 5 mm), and then, the clamp 64B is moved away from the absorbent product so that it is no longer in contact with the absorbent article 2. Before moving the clamp 64B, the absorbent article 2 should not be adhered thereto to avoid dragging of the absorbent article when the sliding clamp 64B is released.

Once the absorbent article 2 is released from the clamp 64B, the absorbent article 2 is allowed to “regain” its original shape for approximately one minute. After one minute has passed, the highest thickness of the absorbent article 2 is measured at its centerline 80, as shown for example, in FIG. 14 by way of a straight edge 84. The straight edge 84 is placed on the top surface of the absorbent article without applying pressure to the sample, so that it can read the thickness of the absorbent article from the clamp ruler 74 placed on the vertical wall 76 of the clamp 64A. The thickness of the straight edge 84 should be about 0.5 mm. The thickness release is recorded for each of thirty samples, and an average thickness release is obtained from the thirty absorbent articles.

Various testing was performed to verify the increased core integrity. In particular, thirty samples of a first inventive product (“Inventive Product 1—Moderate”) were compared to various prior art absorbent articles, each having a Rothwell value of between about 260 g to 399 g. In particular, the Inventive Product 1—Moderate was compared to a product sold under the trademark POISE® Moderate which is manufactured by Kimberly-Clark, Inc. The POISE® Moderate includes a top sheet, back sheet, and an absorbent core therebetween. The absorbent core includes a small core adjacent the back sheet, and a larger core on the top side of the small absorbent core. A highloft material is disposed beneath the top sheet, and a tissue layer is disposed between the big core and high loft material.

Similar comparative testing was performed on several prior art absorbent articles including the TENA® Moderate Regular manufactured by SCA Hygiene Products, and the OPTIONS® Moderate manufactured by First Quality and distributed by Walmart, Inc. Using the core resiliency test described above, the following data was collected. A summary of the results in provided below in Table 1.

TABLE 1

| PRODUCT | THICKNESS RELEASE (mm) |
|---|---|
| INVENTIVE PRODUCT 1- | 5.03 |
| MODERATE | |
| POISE ® MODERATE | 14.83 |
| OPTIONS ® MODERATE | 12.97 |
| TENA ® MODERATE REGULAR | 8.97 |

As demonstrated by the results above, the thickness release of the Inventive Product 1 was significantly lower than the prior art absorbent products, such as POISE® Moderate, OPTIONS® Moderate and TENA® Moderate Regular, demonstrating a significant improvement in product resiliency.

Similar comparative testing was performed on “HEAVY” type products to confirm the increased core resiliency of the inventive absorbent articles described herein. In particular, thirty samples of a second inventive product (“Inventive Product 2—Heavy”) were compared to various prior art absorbent articles. In particular, the Inventive Product 2—Heavy was compared to the POISE® Maximum (manufactured by Kimberly-Clark), the TENA® Heavy Regular (manufactured by SCA Hygiene Products), and the OPTIONS® Maximum (manufactured by First Quality and distributed by Walmart, Inc.). All of the products tested have a Rothwell value of between about 400 g to 600 g. Using the core resiliency test described above, the following data was collected. A summary of the results in provided below in Table 2.

TABLE 2

| PRODUCT | THICKNESS RELEASE (mm) |
|---|---|
| INVENTIVE PRODUCT 2- HEAVY | 7.13 |
| POISE ® MAXIMUM | 14.83 |
| OPTIONS ® MAXIMUM | 19.23 |
| TENA ® HEAVY REGULAR | 12.30 |

As demonstrated by the results above, the thickness release of the Inventive Product 2 was significantly lower than the prior art absorbent products, such as POISE® Maximum, OPTIONS® Maximum, and TENA® Heavy Regular, demonstrating a significant improvement in product resiliency.

### TUT Tumbling Test

A tumbling test is useful for assessing absorbent products with an average Rothwell value range of between about 260 g to 800 g. The equipment used is described in the ISO 12945-1 method, section 5.1. Such a pilling box is available through SDL Atlas, located in Rock Hill, S.C., model M227.

According to the method described therein, all of the internal surfaces of the box are lined with cork jointing material. Calibration and maintenance of pilling box given in Annex A of the ISO 12945-1 method should be followed. The pilling box is rotated about a horizontal axis passing through the centers of two opposites faces. Five polyurethane specimen tubes are used, which function to compress the absorbent article during rotation. The pilling test method according to ISO 12945-1 is a standardized test used throughout the textile industry.

In order to determine whether the absorbent products have maintained their core resiliency, a light box is used to assess cracks in the product. Preferably, the light box should be large enough to provide uniform illumination throughout the length and width of the pad. More preferably, the product should cover less than 40% of the total light surface of the lamp. Such a light box is available through a company called UVP located in Upland, California-Model TW-43.

Prior to administering the tumbling test according to features of the present invention, the specimens should be conditioned to room temperature of about (21+/−2° C.) with a relative humidity of about (65+/−2%) for at least 24 hours. During the preparation stage, the absorbent articles should be removed from their bags and individual wrappings. Once the specimens are properly conditioned, release paper is removed from the specimen (if present) and baby powder is spread over the adhesive to remove its tackiness, and to prevent the specimen from sticking on itself during the compression period.

Before testing the absorbent articles, the absorbent articles should be assessed. If an absorbent article includes a crack, an unpurposeful partial split/break, or is missing a section of its absorbent core, the product should be rejected. In total, ten absorbent articles shall be tested per product variant. The absorbent articles should look identical taking into consideration minor process variations. If there is an obvious difference, the outlier should be rejected and replaced by another sample.

Once the absorbent articles are properly prepared, they are ready for testing. The first step is to determine the center of the product, and to mark it at its center point, as shown in FIG. 15. Like the core resiliency test described above, numerous products should be tested per product variant. These products should look identical taking into consideration minor process variation.

The products are placed on the light box prior to administering the tumbling test. Preferably, there should be a relatively uniform amount of light shinning through the absorbent core. However, it is not uncommon to find small darker areas of pulp in the core. These areas occur as a result of improper defiberization of the pulp in the mill. These small unfiberized clumps will not interfere with the test results. If there is an obvious difference, the outlier should be rejected and replaced by another sample.

The product is placed on a light box with the user side facing the light box's surface for inspection. That is, the specimen should be placed on the light table with the top sheet facing down. The vertical distance between the camera and the light table should be about 300 mm, and the vertical distance between the technician doing the test and the light table should be about 400-800 mm. Because the analysis compares individual products before and after tumbling, process irregularities or design characteristics should be taken into consideration. For example, where a particular concentration exists prior to tumbling, that concentration should be negligible when considering whether there is a crack or failure of the product.

A picture of the sample product should be taken when it is on the light box. After the picture is taken, the sample product is dosed. With reference to FIG. 15, the dosing apparatus 100 is similar to the dosing apparatus 40 described above, and includes a funnel 102 supported on a base support 104. The particular sample is dosed according to its Rothwell value. For example, for products with a Rothwell value of between about 260 g to 399 g, 50 ml of 0.9% saline solution (21+/−2° C.) is used, and for products having an average Rothwell value of between about 400 g to 800 g, 75 ml of 0.9% saline solution (21+/−2° C.) is used.

Similar to the core resiliency test, liquid is dosed onto the absorbent article at a constant slow flow rate. The flow rate may be adjusted based on how slow or fast the product is absorbing the liquid. However, the liquid should not overflow the circular base of the dosing apparatus 100, and the liquid should all be absorbed by the absorbent article in less than 1 minute. Once all the liquid has been absorbed, the dosing apparatus 100 can be removed from the absorbent product 2.

After the product is dosed, 10 minutes is allowed to lapse. The untumbled, dosed product should be place on the light box top sheet facing down and photographed, prior to tumbling. The sample is then placed into the pilling box chamber with five polyurethane tubes. For products that are longer than 235 mm, the products may be placed at an angle or folded onto themselves to fit within the box. The pilling box is set to rotate for 1800 cycles, for about 15-20 minutes. Once the equipment has stopped, the specimen is gently removed and placed on the light box for determining whether the sample has cracked or been damaged.

Core cracks or damage is defined as areas where the absorbent core matrix has ruptured due to the strain of the tumbling. As a result, the absorbent material has severely shifted creating areas of high and low density not caused by the absorption of the test liquid.

The tumbled sample is compared to the picture of the undosed sample to determine if there is a crack by way of obvious differences in light shining through the product.

A total of ten products should be tumbled and compared under the light box. A product fails the tumbling test when at least one sample blows out in the pilling box, or if three or more products have a crack. A product blow out is when the samples' top sheet and back sheet seal is ruptured so that the absorbent material is exposed.

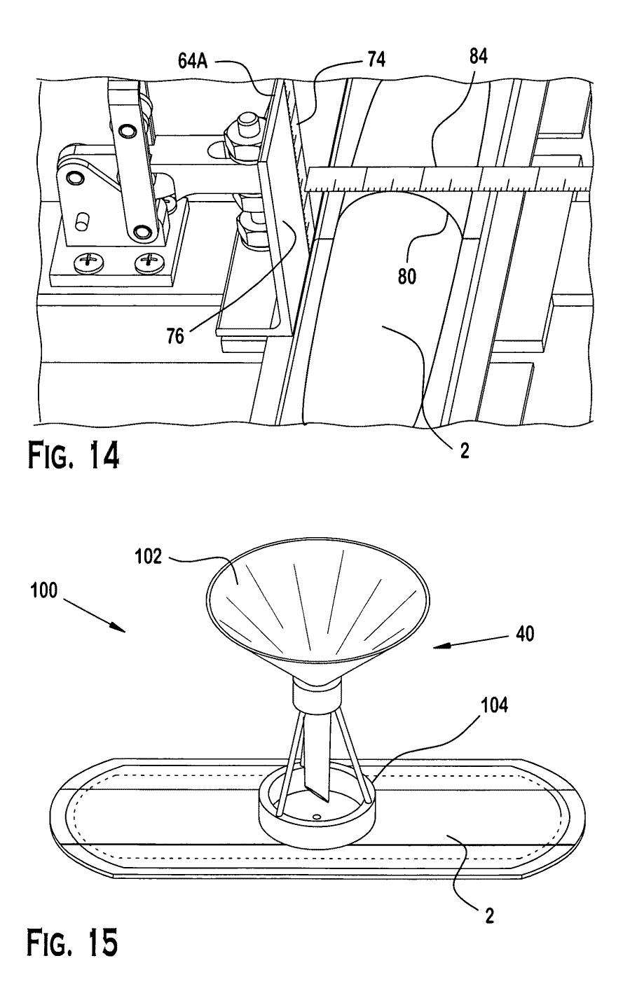

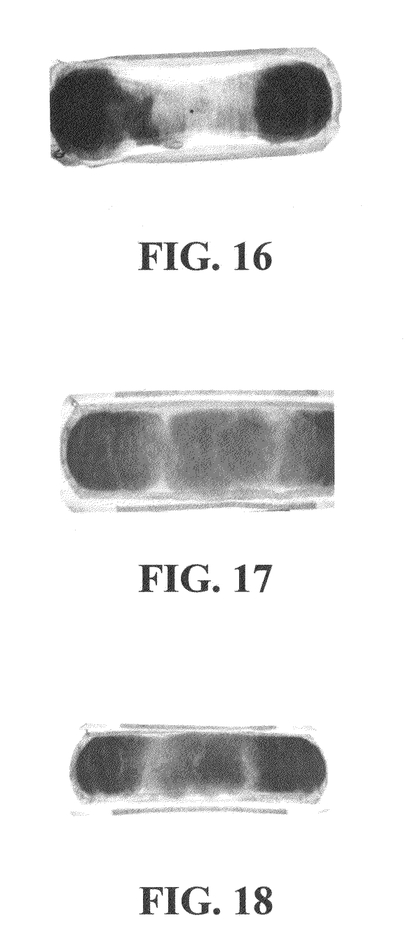

With reference to FIGS. 16-23, photographs of samples with and without cracks are shown. The contrast and the brightness have been changed on all photographs to enhance the possibility to see the cracks. With reference to FIGS. 16-20, each of these samples represent prior art absorbent articles after being run in the pilling box, each of the samples exhibiting “cracks”. FIG. 16 represents a prior art article having a printed top sheet. As shown in FIG. 16, large amounts of the absorbent material fluctuated to ends of the products. However, as observed during the testing, the shaded area in the middle of the product occurred due to the printed top sheet and not due to the presence of absorbent material. This observation is readily determined by the human eye, and not as easily discernible from photographs.

With reference to FIGS. 17 and 18 (representing prior art articles with a printed top sheet and white transfer layer), large amounts of the absorbent material fluctuated to ends of the products. As observed during the testing, the shaded area in the middle of the product occurred due to the printed top sheet and white highloft present, and not due to the presence of absorbent material.

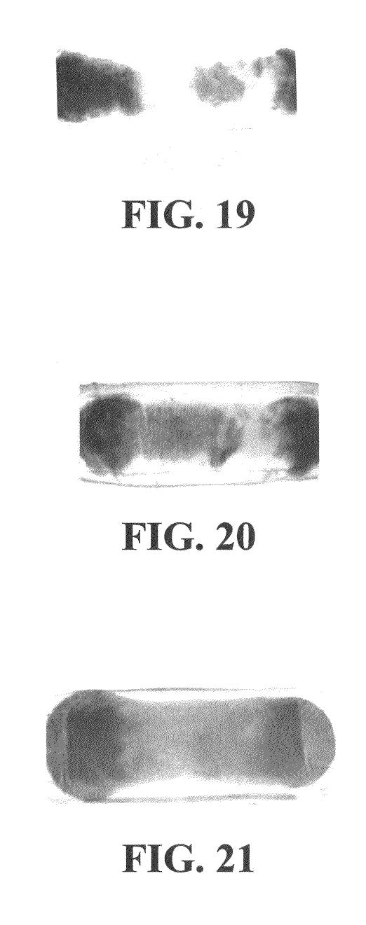

With reference to FIG. 19 (representing prior art article with a white top sheet and white transfer layer), large amounts of the absorbent material fluctuated to ends of the products. As observed during the testing, the shaded area in the middle of the product occurred due to the colored transfer layer and not due to the presence of absorbent material.

With reference to FIG. 20 (representing prior art article with a white top sheet and colored transfer layer, large amounts of the absorbent material fluctuated to ends of the products. However, as observed during the testing, the shaded area observed occurred due to the colored transfer layer and not due to the presence of absorbent material.

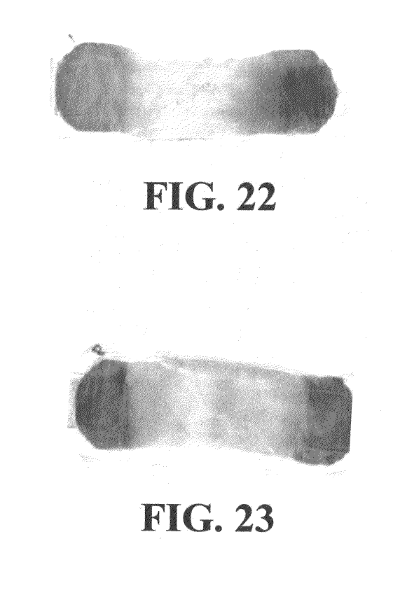

With reference to FIGS. 21-23, the inventive products of the present invention are illustrated. In particular, each of the three products shown in FIGS. 21-23 demonstrated no cracks after being run in the pilling box. However, with reference to FIGS. 22-23, the products shown therein, which are modified to maximize the contrast and has purposeful perforations, have no rupture of the absorbent core matrix, only minor migration of the absorbent material. In particular, there is no defined area where no absorbent material is present so as to impede the wicking of liquid from one end point to another. Accordingly, the product shown in FIGS. 22-23 would be determined to have “no cracks”.

Although photographs have been taken to help demonstrate the presence or absence of cracks, it should be understood that the human eye with normal eye sight and human perception is the best tool to evaluate if there is a crack or not. The determination of a crack or no crack is made by looking at the physical products, and not at photographs. As such, depth perception plays a part in discerning whether there is a crack or not. To further distinguish cracks, a weak colorant may be added to the testing saline solution.

Tests were performed by Specialized Technology Resources, Inc. located in Enfield, Connecticut. Tests were performed on the Inventive Product 1—Moderate and the Inventive Product 1—Heavy, described above. With regard to the Inventive Product 2—Heavy, all ten products tested were found to have no cracks. With regard to the Inventive Product 1—Moderate, all but one of the ten products were found to have no cracks. Only one product was found to have a single crack. The Inventive Product 1—Moderate and the Inventive Product 2—Heavy were the only products to pass the tumbling test described herein. This is due in part to the use of an airlaid absorbent core having a network of bicomponent fibers in the outer layers.

The tumbling test as described in the present invention was also performed on the TENA® Moderate, TENA® Heavy, POISE® Moderate, POISE® Maximum, and OPTIONS® Maximum and OPTIONS® Moderate. For TENA® Moderate, the first two products tested had a single crack and the third sample tested blew out. The testing was stopped due to the product failure. For the TENA® Heavy, every specimen tested had either one or two cracks. For POISE® Moderate, two products had one crack, seven products had two cracks and one product had no cracks. For POISE® Maximum, three specimens had one crack, five specimens had two cracks, one specimen had three cracks, and one specimen had no cracks. For the OPTIONS® Maximum, the first two specimens tested had one crack and a slight blowout. The second two specimens tested had one crack and a big blowout. The testing was then stopped due to the product failure. For the OPTIONS® Moderate, six of the products had a single crack, three of the products had two cracks, and one product blew out. Each of these prior art absorbent articles had three or more products present with cracks, thereby failing the tumbling test.

The core resiliency test and tumbling test confirm that the absorbent article described herein have superior qualities when compared to prior art absorbent articles. This is due in part to the particular make up of the absorbent core. The various layers and materials create a complex shape retention network. As such, an improved liquid handling structure is created that distributes liquid evenly while maintaining its shape/thinness. Further, the even distribution of the liquid handling and addition of bicomponent fibers can result in a product that better withstands stress forces.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

1. An absorbent article, comprising:
a back sheet;
a top sheet; and
an absorbent core therebetween, said absorbent core passing a tumbling test;
wherein the article has a thickness when compressed laterally and released in a core resiliency test of less than 7 mm for an article having a Rothwell value from about 260 to about 399 g.

2. The absorbent article of claim 1, wherein the absorbent core is made of an airlaid material.

3. The absorbent article of claim 2, wherein the airlaid material includes two or more layers.

4. The absorbent article of claim 3, wherein the airlaid material comprises 0-10% by weight of a tissue or nonwoven layer, 20-65% by weight of pulp, 30-60% by weight of a superabsorbent polymer, 0-5% by weight polymer dispersion binder, and 2-10% by weight of bicomponent fibers.

5. The absorbent article of claim 1, wherein the absorbent core is an airlaid having a first layer, a second layer, and a third layer, said second layer being interposed between said first and third layers, said first and third layer being made from pulp and bicomponent fibers, and optionally superabsorbent polymers, the second layer is made from pulp and superabsorbent polymer.

6. The absorbent article of claim 5, wherein only the first, third and an optional fourth layer contain bicomponent fibers, and wherein optionally only the top surface and/or the bottom surface of the airlaid is treated with a polymer dispersion.

7. The absorbent article of claim 1, wherein the absorbent core includes an inner line and an outer line on each side of a longitudinal centerline, each said inner lines and outer lines including a plurality of slits having spaces therebetween.

8. The absorbent article of claim 7, wherein one or both of the outer line and the inner line are shaped as crescents.

9. The absorbent article of claim 1, wherein the absorbent article is an incontinence pad or a sanitary napkin.

10. An absorbent article, comprising:
a back sheet;
a top sheet; and
an absorbent core therebetween, said absorbent core passing a tumbling test;
wherein the article has a thickness when compressed laterally and released in a core resiliency test of less than 11 mm for an article having a Rothwell value from about 400 to about 800 g.

11. The absorbent article of claim 10, wherein the Rothwell value is from about 400 to about 600 g.

12. The absorbent article of claim 10, wherein the absorbent core is made of an airlaid material.

13. The absorbent article of claim 12, wherein the airlaid material includes two or more layers.

14. The absorbent article of claim 13, wherein the airlaid material comprises 0-10% by weight of a tissue or nonwoven layer, 20-65% by weight of pulp, 30-60% by weight of a superabsorbent polymer, 0-5% by weight polymer dispersion binder, and 2-10% by weight of bicomponent fibers.

15. The absorbent article of claim 10, wherein the absorbent core is an airlaid having a first layer, a second layer, and a third layer, said second layer being interposed between said first and third layers, said first and third layer being made from pulp, bicomponent fibers, and optionally superabsorbent polymers, the second layer is made from pulp and superabsorbent polymer.

16. The absorbent article of claim 15, wherein only the first, third and an optional fourth layer contain bicomponent fibers, and wherein optionally only a top surface and/or a bottom surface of the airlaid is treated with a polymer dispersion.

17. The absorbent article of claim 10, wherein the absorbent core includes an inner line and an outer line on each side of a longitudinal centerline, each said inner lines and outer lines including a plurality of slits having spaces therebetween.

18. The absorbent article of claim 17, wherein one or both of the outer line and the inner line are shaped as crescents.

19. The absorbent article of claim 10, wherein the absorbent article is an incontinence pad or a sanitary napkin.

20. An absorbent article, comprising:
a back sheet;
a top sheet; and
an absorbent core therebetween;
wherein said absorbent core is an airlaid material including two or more layers, said absorbent core being made from a superabsorbent polymer, pulp, and bicomponent fibers, wherein the bicomponent fibers are disposed adjacent a top surface and bottom surface of the absorbent core.

21. The absorbent article of claim 20, wherein the absorbent core is an airlaid having a first layer, a second layer, and a third layer, said second layer being interposed between said first and third layers, said first and third layer being made from pulp, bicomponent fibers, and optionally superabsorbent polymers, the second layer is made from pulp and superabsorbent polymer.

22. The absorbent article of claim 21, wherein only the first, third and an optional fourth layer contain bicomponent fibers, and wherein optionally only the top surface and/or the bottom surface of the airlaid is treated with a polymer dispersion binder.

23. The absorbent article of claim 20, wherein the airlaid material comprises from 0-10% by weight of a tissue or nonwoven layer, 20-65% by weight of pulp, 30-60% by weight of a superabsorbent polymer, 0-5% by weight polymer dispersion binder, and 2-10% by weight of bicomponent fibers.

24. The absorbent article of claim 20, wherein the absorbent core includes an inner line and an outer line on each side of a longitudinal centerline, each said inner lines and outer lines including a plurality of slits having spaces therebetween.

25. The absorbent article of claim 24, wherein one or both of the outer line and the inner line are shaped as crescents.

26. The absorbent article of claim 20, wherein the absorbent article is an incontinence pad or a sanitary napkin.
